(19) United States
(12) Patent Application Publication
(10) Pub. No.: US 2025/0017735 A1
(43) Pub. Date: Jan. 16, 2025

(54) NON-IMPINGING DUAL MOBILITY HIP PROSTHESIS

(71) Applicant: SIGNATURE ORTHOPAEDICS EUROPE LTD, Dublin (IE)

(72) Inventors: Declan BRAZIL, Lane Cove West (AU); Christopher BURGESS, Dublin (IE)

(21) Appl. No.: 18/714,358

(22) PCT Filed: Dec. 16, 2022
(86) PCT No.: PCT/AU2022/051526

§ 371 Date: May 29, 2024

(30) Foreign Application Priority Data
Dec. 17, 2021 (AU) 2021904103

(51) Int. Cl. A61F 2/34; A61F 2/32; A61F 2/36

(52) U.S. Cl. CPC A61F 2/34; A61F 2002/3208; A61F 2002/3233; A61F 2002/3403; A61F 2002/3435; A61F 2002/3443; A61F 2002/3611; A61F 2002/3625

(57) ABSTRACT

Dual mobility hip prosthesis has a liner rim that recessed with respect to an acetabular cup rim thereof so that an adjacent contact surface of the neck impinges the acetabular cup rim at extreme positions without impinging the liner rim. Furthermore, the acetabular cup rim defines an inner contact face correspondingly angled to the adjacent contact surface of the neck at the extreme positions to reduce point contact loading between the acetabular cup rim and the adjacent contact surface of the neck. As such the present prosthesis tolerates prosthetic impingement between the rim of the acetabular cup and the neck of the femoral component by mitigating against point contact loading force whilst eliminating impingement of edges of the polymeric liner between an edge of the neck and the rim of the acetabular cup and also allowing for sufficiently deep acetabular componentry with reduced likelihood of dislocation.

### FIELD OF THE INVENTION

This invention relates to a type of dual mobility hip prosthesis.

### BACKGROUND OF THE INVENTION

Hip arthroplasty involves surgical procedures to replace damaged parts of a human hip joint with artificial hip prosthesis.

Such hip prosthesis typically comprises acetabular componentry to engage the pelvic acetabulum and femoral componentry attached to the femur (thighbone).

The femoral componentry articulates within the acetabular componentry to mimic the “ball-and-socket” of a healthy human hip joint.

The acetabular componentry typically comprises a metal acetabular cup which is cemented, screwed or otherwise affixed to the acetabulum. A polymeric liner may be permanently affixed within the acetabular cup to defines a smooth articular inner bearing surface for the femoral componentry.

The femoral componentry typically comprises a stem which is inserted into a resected intermedullary femoral canal of the thigh bone. The stem terminates in a superior angled neck which engages a spherical femoral head at a distal end thereof, typically by way of conical interlock. The femoral head articulates within the acetabular componentry.

Dislocation is a medical condition when the femoral head is forced out of the cavity defined by the acetabular componentry. Apart from causing functional impairment, instability and pain, dislocation may require revision surgery.

Dislocation may occur at extreme positions of the femoral componentry with respect to the acetabular componentry wherein the neck impinges the acetabular componentry to cantilever the femoral head out of the socket defined by the acetabular component.

The point of impingement of the neck against the rim of the acetabular cup acts as a fulcrum and the relative short length of the neck from the fulcrum to the femoral head centroid with respect to the length of the femur acts as a relatively short lever which induces high leverage force which can lift the femoral head out of the socket defined by the acetabular componentry. Eventually, the femoral head centroid moves above the perimeter of the acetabular componentry socket, causing dislocation.

Attempts to decrease likelihood of dislocation involve making the acetabular componentry “deeper”. However, making the acetabular componentry deeper increases the likelihood of damage to the liner wherein the lateral forces (i.e. forces radially perpendicular to a polar axis of the acetabular cup) impinge the liner between the neck and inner surfaces of the acetabular cup, causing damage to the liner.

Damage to the liner may be mitigated by having a shallower acetabular componentry to eliminate lateral forces impinging the liner between the acetabular cup and the neck. For example, acetabular componentry may be sufficiently shallow so that a contacting edge of the neck is orthogonal (taking into account the width of the neck) so that the neck lies completely flat across the rim on the acetabular componentry, thereby not applying lateral impinging forces to the liner.

However, this approach results in shallower acetabular componentry, thereby increasing the likelihood of dislocation.

As such, there is essentially a trade-off between mitigating liner impingement and risk of dislocation.

To avoid prosthetic impingement, hip prosthesis may be designed with a much higher head-to-neck diameter ratio as compared to that occurring in the native hip. However, prosthetic impingement may yet occur.

Dual mobility prostheses involve the interposition of an extra and intermediate dual mobility bearing component. The effect of a dual mobility bearing component is to improve (i.e., increase) the effective head-to-neck diameter ratio and so further avoid prosthetic impingement.

However, insertion depth is especially important for dual mobility prosthesis to reduce likelihood of dislocation given the greater range of motion offered thereby and the prosthesis having dual bearing interfaces.

To address issues of liner damage from prosthetic impingement, WO 2017/214095 A1 (Holiday, et al.) 14 Dec. 2017 proposes a solution which has “deep” acetabular componentry but wherein the edge of the liner is protected with a bumper formation.

Specifically, Holiday et al. proposes a flexible bumper configured to extend from the acetabular component and prevent contact between the femoral neck and the acetabular component.

The present invention seeks to provide a way which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein hip prosthesis designed to address the aforedescribed problems of mitigating dislocation potential and acetabular cup liner damage and which is applied to dual mobility hip prosthesis.

Specifically, there is provided dual mobility hip prosthesis comprising an acetabular cup defining an outer acetabulum fixing surface and an inner fixing surface.

The prosthesis further comprises a fixed liner defining an outer fixing surface configured for fixing to the inner fixing surface of the acetabular cup and an inner articular surface.

The prosthesis further comprises a bearing component defining an outer articular surface configured for articulating against the inner articular surface of the fixed liner and an inner articular surface.

The prosthesis further comprises a femoral component comprising a neck engaging a head at a distal end of the neck, the head defining an exterior articular surface configured for articulating against the inner articular surface of the bearing component

The acetabular cup defines an acetabular cup rim and the liner defines liner rim.

The present prosthesis is configured so that the liner rim is recessed with respect to the acetabular cup rim so that an adjacent contact surface of the neck impinges the acetabular cup rim directly at extreme positions of the femoral component with respect to the acetabular cup without impinging the liner rim.

Furthermore, the acetabular cup rim defines an inner contact face correspondingly angled to the adjacent contact surface of the neck at the extreme positions to reduce point contact loading between the acetabular cup rim and the adjacent contact surface of the neck.

As such, in contradistinction to prior art hip prosthesis which avoid direct impingement of the neck on the rim of the acetabular cup (including Holiday et al. which teaches reinforcing the acetabular cup polymeric liner with bumper formations), the present arrangement is designed to tolerate prosthetic impingement between the rim of the acetabular cup and the neck of the femoral component, thereby eliminating impingement of edges of the polymeric liner between an edge of the neck and the rim of the acetabular cup.

Furthermore, the present hip prosthesis is designed to tolerate prosthetic impingement wherein the acetabular cup rim is designed to mitigate against point contact loading forces between the acetabular cup rim and neck which could fracture the neck.

As such, the present dual mobility hip prosthesis allows for a “deeper” construction to mitigate against dislocation, and which mitigates prosthetic impingement issues without using bumper formations and the like as are recommended by prior art arrangements.

Furthermore, the liner may be non-rotatably captured by the inner fixing surface of the acetabular cup. The liner may be non-rotatably captured by the acetabular cup without insertion of a locking ring, which allows the liner rim to lie close to the inner contact face to thereby maximise coverage of the acetabular cup by the liner.

The liner may comprise a bung protruding at a polar region thereof which inserts into a placement socket of the acetabular cup. Furthermore, corresponding equatorial peripheries of the acetabular cup and the liner comprise respective non-rotatably engaging frustoconical locking tapers.

The bung and the locking tapers may non-rotatably engage the liner within the acetabular cup, avoiding disadvantages of locking rings and the like.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a disassembled perspective view of an acetabular cup and liner therefore of dual mobility hip prosthesis;

FIG. 2 shows an assembled perspective view of the acetabular cup and liner;

FIG. 3 shows a magnified view of a rim of the acetabular cup;

FIG. 4 shows a cross-sectional view of the acetabular cup engaging the liner;

FIG. 5 shows a cross-sectional view of the liner;

FIG. 6 shows a side view of the liner;

FIG. 7 shows a cross-sectional view of the dual mobility hip prosthesis;

FIG. 8 shows a top view of the dual mobility hip prosthesis;

FIG. 9 shows a magnified side cross-sectional view illustrating the interface; and of the rim of the acetabular cup and the rim of the liner; and

FIG. 10 shows a magnified cross-sectional view illustrating interfacing of the acetabular componentry with femoral componentry of the present prosthesis.

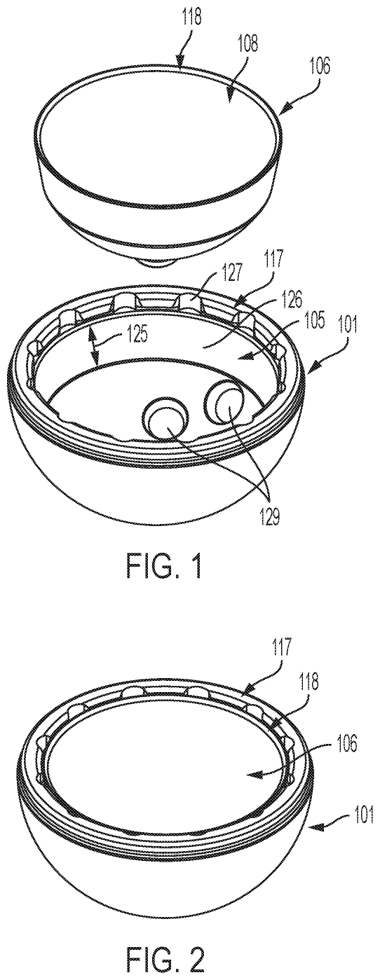

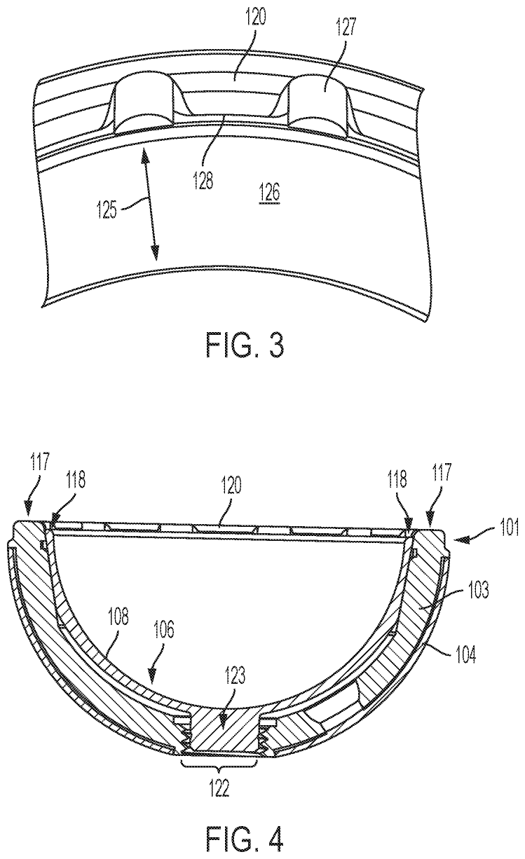

### DESCRIPTION OF EMBODIMENTS

Dual mobility hip prosthesis 100 comprises an acetabular cup 101 defining an outer acetabulum fixing surface 102 which is affixed using cement, bone screws or the like to the pelvic acetabulum. The acetabular cup 101 may comprise a structural body 103 and an exterior biocompatible and/or osteointegration shell 104. The acetabular cup 101 may comprise bone fixing screw apertures 129.

The acetabular cup 101 is typically metallic and may comprise titanium.

The acetabular cup 101 further defines an inner fixing surface 105.

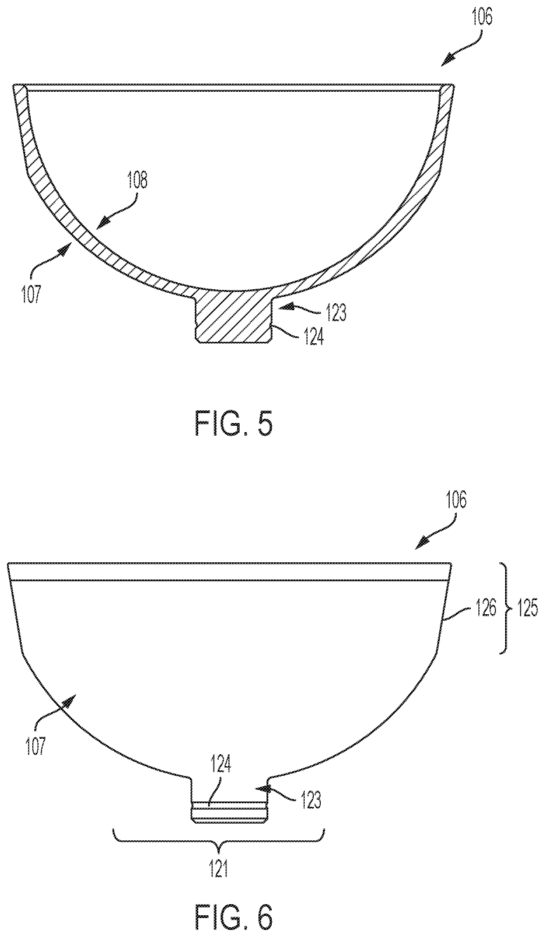

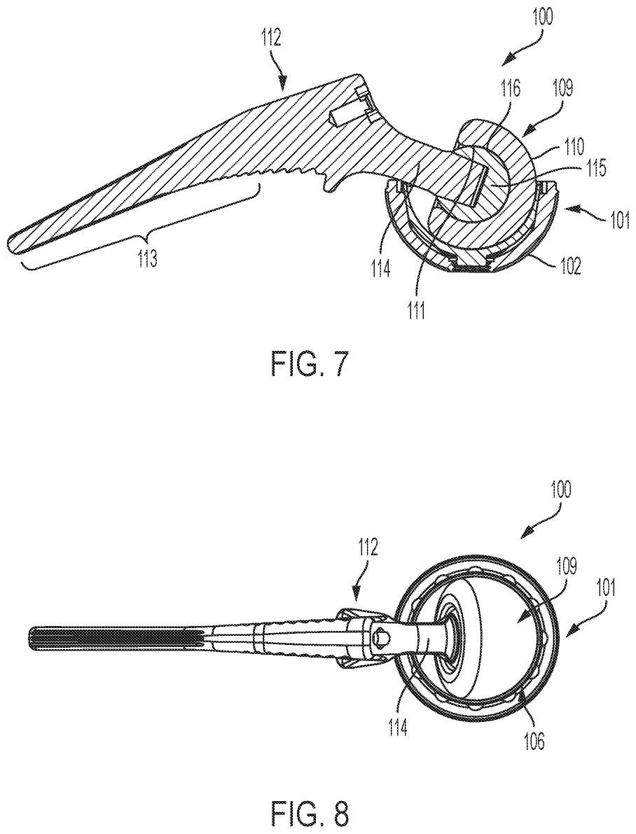

The prosthesis 100 further comprises a fixed liner 106 defining an outer fixing surface 107 configured for fixing to the inner fixing surface 105 of the acetabular cup 101. The fixed liner 106 further defines an inner articular surface 108. With reference to FIG. 5, the inner articular surface 108 may be semi-spherical.

The fixed liner 106 is typically polymeric and may comprise High-density polyethylene (HDPE), Ultra-high-molecular-weight polyethylene (UHMWPE) or the like.

The prosthesis 100 further comprises a bipolar bearing component 109 which defines an outer articular surface 110 configured for articulating against the inner articular surface 108 of the fixed liner 106. The bearing component 109 further defines an inner articular surface 111. The bearing component 109 may similarly be polymeric.

The prosthesis 100 comprises a femoral component 112 having a stem 113 for insertion in the intermedullary canal of the femur and a neck 114 engaging a spherical head 115 at a distal end thereof. The head 115 may define a frustoconically tapered blind bore into which a correspondingly frustoconical profile of the neck 114 is frictionally engaged.

The head 115 defines an exterior articular surface 116 configured for articulating against the inner articular surface 111 of the bearing component 109.

The acetabular cup 101 defines an acetabular cup rim 117 and the liner 106 defines a liner rim 118.

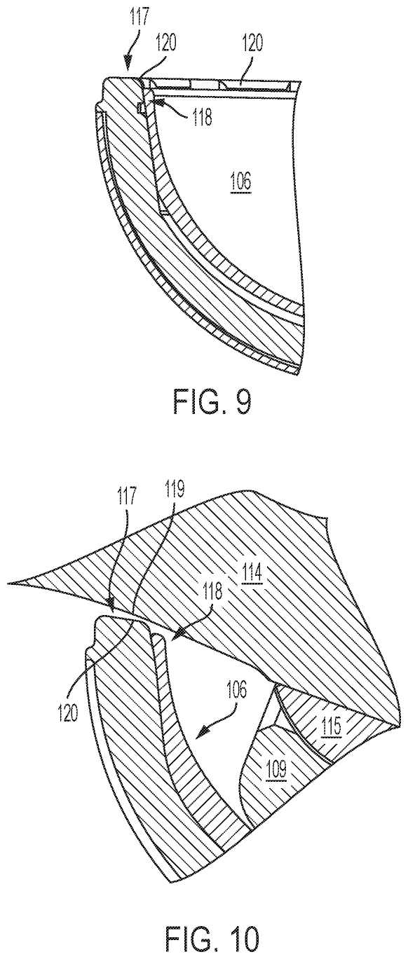

As is apparent from FIG. 10, the liner rim 118 is recessed with respect to the acetabular cup rim 117 so that an adjacent contact surface 119 of the neck 114 impinges the acetabular cup rim 117 at extreme positions of the femoral component 112 with respect to the acetabular cup 101 without impinging the liner rim 118.

Furthermore, the acetabular cup rim 117 defines an inner contact face 120 correspondingly angled to the adjacent contact surface 119 of the neck 114 at the extreme positions to reduce point contact loading force between the acetabular cup rim 117 and the adjacent contact surface 119 of the neck 114.

In the embodiment shown in FIG. 10, the inner profile of the acetabular rim 117 may be chamfered so that the inner contact face 120 is flat. In this way, the inner contact face 120 is designed to flatly contact against the corresponding cross-sectional flat edge of the adjacent contact surface 119 of the neck 114.

The contact face 120 may be sufficiently wide and, for example, may have a width of greater than 2 mm.

The inwardly angled contact face 119 may allow for relatively deep acetabular componentry. As is evident from FIG. 4, the liner 106 may have a cross-section having an arc angle of greater than 170°, preferably up to approximately 180°. Despite the relative depth of the liner 106, the neck 114 does not impinge the rim 118 of the liner 106.

Furthermore, this arrangement maximises the coverage extent of the liner wherein, as is further shown in FIG. 10, the rim 118 of the liner 106 may lie quite close to the inner contact face 120, thereby covering most of the entirety of the inner articular surface 105 of the acetabular cup 101. For example, the rim 118 of the liner 106 may lie within approximately 2 mm of the inner contact face 120.

In the embodiment shown in FIG. 10, the liner 106 may be captured by the acetabular cup 101 without insertion of an intervening locking ring. Furthermore, avoiding use of a locking ring allows the liner rim 118 to lie very close to the angled contact face 120.

The liner 106 may be exteriorly captured by the interior fixing surface 105 of the acetabular cup 101 to hold the liner 106 in place without insertion of a locking ring.

The liner 106 may be exteriorly captured by the acetabular cup 101 at a polar region 121 thereof. Specifically, as is shown in FIG. 4, the acetabular cup 101 may define a polar socket 122. The socket 122 may be threaded. During insertion of the acetabular cup 101 a distally threaded rod of an insertion tool may engage the threaded polar socket 122 of the acetabular cup 101 to aid placement thereof.

The liner 106 may comprise an integrally formed bung 123 protruding from the polar region 121 thereof which engages the polar socket 122 of the acetabular cup 121. The bung 123 may be sized to frictionally engage the socket 122.

The bung 123 may comprise exterior circumferential formations 124 which interfere with thread of the polar socket 122.

Furthermore, corresponding equatorial peripheries 125 of the acetabular cup 101 and the liner 106 may comprise non-rotatably engaging frustoconical locking tapers 126. These tapers 126 complement the securement of the bung 123 so that the liner 106 is held securely both at polar 121 and equatorial 125 regions thereof.

The acetabular cup 101 may comprise inner anti-torsion recesses 127 and the liner 106 may comprise outer anti-torsion protrusions (not shown) which correspondingly plug into the anti-torsion recesses 127.

In embodiments, the liner 106 is coated with Diamond-like carbon (DLC) which may reduce fretting corrosion, especially on the tapers of the liner 106.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. Dual mobility hip prosthesis comprising:
an acetabular cup defining an outer acetabulum fixing surface and an inner fixing surface;
a fixed liner defining an outer fixing surface configured for fixing to the inner fixing surface of the acetabular cup and an inner articular surface;
a bearing component defining an outer articular surface configured for articulating against the inner articular surface of the fixed liner and an inner articular surface;
a femoral component comprising a neck engaging a head at a distal end of the neck, the head defining an exterior articular surface configured for articulating against the inner articular surface of the bearing component, wherein:
the acetabular cup defines an acetabular cup rim;
the liner defines liner rim;
the liner rim is recessed with respect to the acetabular cup rim so that an adjacent contact surface of the neck impinges the acetabular cup rim at extreme positions of the femoral component with respect to the acetabular cup without impinging the liner rim;
the acetabular cup rim defines an inner contact face correspondingly angled to the adjacent contact surface of the neck at the extreme positions to reduce point contact loading between the acetabular cup rim and the adjacent contact surface of the neck.

2. The prosthesis as claimed in claim 1, wherein the inner contact face is flat.

3. The prosthesis as claimed in claim 2, wherein the inner contact face has a width of greater than 2 mm.

4. The prosthesis as claimed in claim 1, wherein the liner has a cross-sectional arc angle of greater than 170°.

5. The prosthesis as claimed in claim 1, wherein the liner has a cross-sectional arc angle of approximately 180°.

6. The prosthesis as claimed in claim 1, wherein the liner rim lies close to the inner contact face.

7. The prosthesis as claimed in claim 6, wherein the liner rim lies within 2 mm of the inner contact face.

8. The prosthesis as claimed in claim 1, wherein the liner is non-rotatably captured by the inner fixing surface of the acetabular cup.

9. The prosthesis as claimed in claim 8, wherein the liner is non-rotatably captured by the inner fixing surface of the acetabular cup without insertion of an intervening locking ring.

10. The prosthesis as claimed in claim 8, wherein the liner exteriorly engages the acetabular cup at a polar region of the liner.

11. The prosthesis as claimed in claim 10, wherein the acetabular cup comprises a polar socket and wherein the liner comprises an integrally formed bung protruding from the polar region which inserts into the polar socket.

12. The prosthesis as claimed in claim 11, wherein the bung is configured for frictional engagement with the polar socket.

13. The prosthesis as claimed in claim 12, wherein the polar socket is threaded and wherein the bung comprises circumferential formations thereon which interfere with thread of the polar socket.

14. The prosthesis as claimed in claim 11, wherein corresponding equatorial peripheries of the acetabular cup and the liner comprises a non-rotatably engaging frustoconical locking tapers.

15. The prosthesis as claimed in claim 1, wherein the outer fixing surface of the liner comprises an DLC coating.

16. The prosthesis as claimed in claim 14, wherein the outer fixing surface of the liner comprises an DLC coating at the tapers.

17. The prosthesis as claimed in claim 1, wherein the acetabular cup rim defines inner anti-torsion recesses and wherein the liner rim comprises outer anti-torsion protrusions which correspondingly plug into the anti-torsion recesses.
